(19) United States
(12) Patent Application Publication
(10) Pub. No.: US 2023/0144683 A1
(43) Pub. Date: May 11, 2023

(54) PLATFORM AND METHOD FOR DETERMINING CRITICAL TRANSCRIPTION FACTORS (TF) FOR TF-BASED HUMAN INDUCED PLURIPOTENT STEM CELL (HIPSC) DIFFERENTIATION

(71) Applicant: Gameto, Inc, New York, NY (US)

(72) Inventors: Pranam Deb Chatterjee, Cambridge, MA (US); Christian Kramme, New York, NY (US)

(73) Assignee: Gameto, Inc, New York, NY (US)

(21) Appl. No.: 17/981,881

(22) Filed: Nov. 7, 2022

Related U.S. Application Data
(60) Provisional application No. 63/277,292, filed on Nov. 9, 2021.

(51) Int. Cl. G16B 25/10; G06N 20/00; G16B 40/20

(52) U.S. Cl. CPC G16B 25/10; G06N 20/00; G16B 40/20; G06N 3/0454

(57) ABSTRACT

A platform and method for determining critical transcription factors for TF-based hiPSC differentiation. The platform including: a transcriptomic dataset database; at least a processor; a memory communicatively connected to the processor, the memory containing instructions configuring the at least a processor to generate gene regulatory networks from transcriptomic datasets; determine a candidate transcription factor; analyze an impact of the candidate transcription factor in germline cell development; and output a set of critical transcription factors.

### CROSS-REFERENCE TO RELATED APPLICATIONS

This application claims the benefit of priority of U.S. Provisional Patent Application Ser. No. 63/277,292, filed on Nov. 9, 2021, and titled “IDENTIFICATION, INTERROGATION, AND INDUCTION OF CRITICAL TRANSCRIPTION FACTORS FOR IN VITRO GERM CELL DIFFERENTIATION,” which is incorporated by reference herein in its entirety.

### FIELD OF THE INVENTION

The present invention generally relates to the field of in vitro cell differentiation. In particular, the present invention is directed to a platform and method for determining critical transcription factors for TF-based hiPSC differentiation.

### BACKGROUND

One of the most difficult and challenging barriers to having in vitro fertilization be accessible and streamlined is the cumbersome and invasive process of egg retrieval, which often relies on the artificial stimulation of ovulation prior to retrieval, followed by surgical vaginal extraction. Even after these processes, egg retrieval can fail, due to, for example, the lack of follicular production, inadequacy of the eggs retrieved, or inadequate fertilization. Thus, the differentiation of human germ cells, ovarian support cells, neural cells, and the like from readily available pluripotent cells, such as, for example, induced pluripotent stem cells (iPSCs), can not only provide a robust method to streamline the in vitro fertilization process, but can also provide an opportunity to study human reproductive processes at scale.

### SUMMARY OF THE DISCLOSURE

In an aspect, a platform for determining critical transcription factors for TF-based hiPSC differentiation, the platform including: a transcriptomic dataset database; at least a processor; a memory communicatively connected to the processor, the memory containing instructions configuring the at least a processor to generate gene regulatory networks from transcriptomic datasets; determine a candidate transcription factor; analyze an impact of the candidate transcription factor in germline cell development; and output a set of critical transcription factors.

In another aspect, a method for determining critical transcription factors for TF-based hiPSC differentiation, the method including: curating, using a computing device, a transcriptomic dataset database; generating, using the computing device, gene regulatory networks from transcriptomic datasets; determining, using the computing device, a candidate transcription factor; analyzing, using the computing device, an impact of the candidate transcription factor in germline cell development; and outputting, using the computing device, a set of critical transcription factors.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary embodiment of a platform for determining critical transcription factors for TF-based hiPSC differentiation;

FIG. 2A is an exemplary embodiment of a metric calculation method including a differentially expressed gene (DEG) network analysis (DEGA);

FIG. 2B is an exemplary embodiment of a prediction of central TFs in known differentiation protocols using graph theory-based TF discovery pipeline;

FIG. 3 is a schematic diagram illustrating a graph theory-based TF discovery pipeline using a GRN centrality analytic algorithm;

FIG. 4A is a schematic diagram illustrating a 2D monolayer screening format for TF-assisted hPGCLC and oogonia-like formation;

FIG. 4B is an exemplary graph illustrating individual induction of 47 computationally predicted TFs in hiPSCs during monolayer hPGCLC formation in the presence or absence of 1 μg/ml doxycycline;

FIG. 4C is an exemplary bar graph illustrating combinatorial TF induction in the monolayer protocol in the presence or absence of 1 μg/ml doxycycline;

FIG. 4D is an exemplary graph illustrating hiPSCs were induced in triplicate using the monolayer format for hPGCLC formation and DDX4-tdTomato expression was assessed via flow cytometry;

FIG. 4E is an exemplary bar graph illustrating combinations of TFs were induced in triplicate using the monolayer format and assessed for DDX4-tdTomato expression and NPM2-mGreenLantern expression;

FIG. 5 an exemplary embodiment of a machine-learning module;

FIG. 6 an exemplary embodiment of neural network;

FIG. 7 is a diagram of an exemplary embodiment of a node of a neural network;

FIG. 8 is an exemplary flow diagram of a method for determining critical transcription factors for TF-based hiPSC differentiation; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to platform and methods for determining critical transcription factors for TF-based hiPSC differentiation.

Aspects of the present disclosure can be used to enable computer algorithms to predict key regulatory transcription factors involved in the process of germ cell specification and induction and utilizes novel screening technologies to interrogate candidate factors and subsequently select properly differentiated cell-types for functional assessment.

Aspects of the present disclosure allow for curation of databases of transcriptomic datasets from previous studies on differentiation to infer regulatory networks of transcription factors, and establishment of a transcription factor over expression screening platform on iPSCs for targeted differentiation provided a set of candidate transcription factors and readouts of cell state; transcription factor over expression may be performed, without limitation, using CRISPR and/or cDNA approaches. Exemplary embodiments illustrating aspects of the present disclosure are described below in the context of several specific examples.

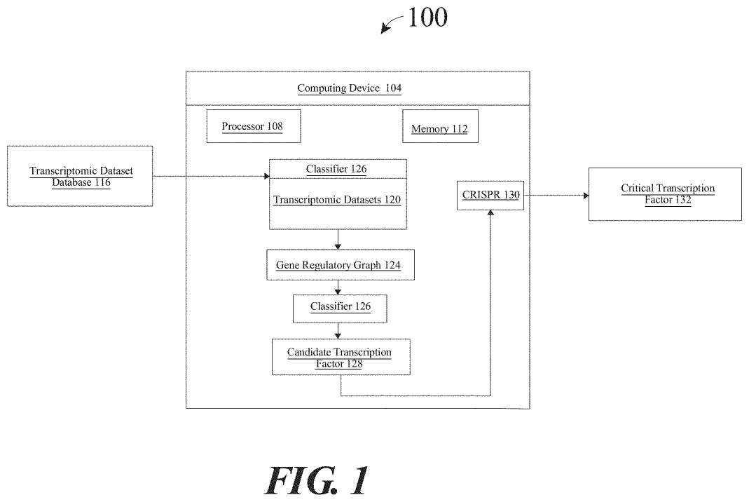

Referring now to FIG. 1, an exemplary embodiment of a platform 100 for determining critical transcription factors 128 for TF-based hiPSC differentiation such as in vitro germ cell differentiation is illustrated. “In vitro,” as used in this disclosure, is a process performed or taking place outside a living organism. As non-limiting examples, in a test tube, culture dish, and the like. A “germ cell,” as used in this disclosure, is any biological cell that gives rise to the gametes of an organism that reproduces sexually. Germ cells differentiate to produce male and female gametes, sperm and unfertilized eggs (oocytes or ova). Germ cells are responsible for the transfer of genetic information to offspring in species with sexual reproduction such as mammals. Germ cell development is dependent on the regulators of gene expression that function at multiple levels, including transcription factors that orchestrate expression at the transcriptional level by binding to enhancer or promoter regions of target genes. Following embryonic genome activation, a series of transcription factors sequentially regulates the activity of a host of genes involved in cell fate decisions, including primordial germ cell specification and migration, sex determination, meiosis and germ cell maturation. Concurrently, developmentally regulated protein expression is also proceeding with coordination by RNA-binding proteins, beginning at fertilization with the translation of maternally inherited mRNA and continuing throughout germ cell development, as evidenced by the number of RNA-binding proteins defined as markers of late stages of germ cell lineages. Moreover, in order to reinforce or redirect cell fate in vitro, it is transcription factors that are most frequently induced, over-expressed or activated. In some embodiments platform 100 may be utilized to identify candidate transcription factors 128 involved in germline cell development. Platform 100 may be used in the differentiation of human germ cells from readily available pluripotent cells, such as, for example, induced pluripotent stem cells (iPSCs). “Pluripotent stem cells,” as used in this disclosure, are cells that are able to self-renew by dividing and developing into the three primary groups of cells that make up a human body, including ectoderm, giving rise to the skin and nervous system; endoderm, forming the gastrointestinal and respiratory tracts, endocrine glands, liver, and pancreas; and mesoderm, forming bone, cartilage, most of the circulatory system, muscles, connective tissue, and more. Pluripotent stem cells may be able to make cells from all three of these basic body layers, so they can potentially produce any cell or tissue the body needs to repair itself. Pluripotent stem cells may include induced pluripotent stem cells (iPSCs), which are derived from skin or blood cells that have been reprogrammed back into an embryonic-like pluripotent state that may enable the development of an unlimited source of any type of human cell needed for therapeutic purposes. For example, iPSC can be prodded into becoming beta islet cells to treat diabetes, blood cells to create new blood free of cancer cells for a leukemia patient, or neurons to treat neurological disorders. Induced pluripotent cells may be derived from embryos, embryonic stem cells made by somatic cell nuclear transfer (ntESCs) and/or an embryonic stem cell from an unfertilized egg. In an embodiment, a pluripotent cell may include a human pluripotent cell. In an embodiment, a pluripotent cell may include an embryonic stem cell, such as a human embryonic stem cell. An “embryonic stem cell,” as used in this disclosure, is a pluripotent stem cell made using embryos or eggs. An embryonic stem cell may include but is not limited to a true embryonic stem cell, a nuclear transfer embryonic stem cell, and/or a parthenogenetic embryonic stem cell. In an embodiment, a pluripotent stem cell may include an induced pluripotent stem cell such as a human induced pluripotent stem cell. A human induced pluripotent stem cell may be derived from skin or blood cells that may be engineered back into an embryonic-like pluripotent state that enables the development of an unlimited source of any type of human cells.

Still referring to FIG. 1, in some embodiments, platform 100 may be used in conjunction with in vitro fertilization (IVF) methods followed by preimplantation genetic diagnosis (PGD) to identify key regulatory transcription factors involved in the process of germ cell specification and induction. “In vitro fertilization,” as used in this disclosure, is a process of fertilization where an egg is combined with sperm in vitro. “Preimplantation genetic diagnosis,” as used in this disclosure, is the genetic profiling of embryos prior to implantation (as a form of embryo profiling). This may include the genetic profiling of oocytes prior to fertilization. An “oocyte,” as used in this disclosure, is a reproductive cell originating in an ovary. An oocyte may include but is not limited to an immature oocyte, a mature oocyte, a group of one or more oocytes, a group of one or more cells, a cumulus oocyte complex and the like. A “cumulus oocyte complex,” as used in this disclosure, is an oocyte containing one or more surrounding cumulus cells. A COC may contain an immature oocyte. A COC may contain a mature oocyte. An “immature oocyte” as used in this disclosure is one or more immature reproductive cells originating in the ovaries. In some embodiments, an immature oocyte may be an oocyte including but not limited to germinal vesicle (GV) and Metaphase 1 (M1) oocytes, as described further below. In some embodiments, an immature oocyte may be a plurality of oocytes. An immature oocyte may be immature cumulus-oocyte-complexes (COCs) taken from a patient. A “mature oocyte” as used in this disclosure, is one or more mature reproductive cells originating in the ovaries. PGD is considered in a similar fashion to prenatal diagnosis. When used to screen for a specific genetic disease, its main advantage is that it avoids selective abortion, as the method makes it highly likely that the baby will be free of the disease under consideration. PGD thus is an adjunct to assisted reproductive technology and requires in vitro fertilization (IVF) to obtain oocytes or embryos for evaluation. Embryos may be generally obtained through blastomere or blastocyst biopsy.

Platform 100 includes a transcriptomic dataset database 116. A “transcriptomic dataset database,” as used in this disclosure is a data structure containing analytical data pertaining to transcriptomes. A “transcriptomic dataset,” as used in this disclosure, is a collection of data related to RNA transcripts. An “RNA transcript,” as used in this disclosure, is the RNA strand that is produced when a gene is transcribed. Precursor mRNA (pre-RNA) is one type of RNA transcript. Pre-mRNA is processed into mature mRNA which in turn is translated into a protein. In some embodiments, transcriptomic datasets 120 may be derived from previous studies on early human germline cell development. To date, there are nearly 200,000 publicly available RNA-seq samples, along with increasing number of genomic and proteomics datasets as well. One example of an RNA-seq data set may include single cell RNA-seq data on samples within various stages of oogenesis. “RNA-seq data,” data as used in this disclosure, is data generated by high-throughput sequencing methods to provide insight into the transcriptome of a cell. Beyond quantifying gene expression, the data generated by RNA-Seq facilitates the discovery of novel transcripts, identification of alternatively spliced genes, and detection of allele-specific expression. “Oogenesis,” as used in this disclosure, is the process of the production of egg cells that takes places in the ovaries. It includes the differentiation of the ovum (egg cell) into a cell competent to further develop when fertilized and is developed from the primary oocyte by maturation. In some embodiments, platform 100 may provide avenues for data analysis and visualization applications pertaining to cells going undergoing oogenesis. In some embodiments, transcriptomic dataset database 116 may be curated using a computing device 104, as described further below, to generate an integrated normalized database comprising RNA-seq data. “Database normalization,” as used in this disclosure, is the process of structuring a relational database in accordance with a series of so-called normal forms in order to reduce data redundancy and improve data integrity. Normal forms may include First Normal Form (1 NF), Second Normal Form (2 NF), Third Normal Form (3 NF), Boyce Codd Normal Form or Fourth Normal Form (BCNF or 4 NF), Fifth Normal Form (5 NF), or Sixth Normal Form (6 NF). “Database integration,” as used in this disclosure, is a process that aggregates information from multiple sources. This may include On-Premises Database Integration, Cloud Database Integration, Hybrid Database Integration, and the like. RNA-seq data may be collected to generate a transcriptomic dataset database 116, annotated by study, cell-type, and experimental details. Transcriptomic dataset database 116 may enable direct access for model training and algorithmic development. In some embodiments, transcriptomic dataset database 116 may be expanded to automatically import, normalize, and curate RNA-seq data from differing cell types. Cell types may include ovarian cells and/or reproductive cells as disclosed in U.S. Nonprovisional application Ser. No. 17/941,423, filed on Sep. 9, 2022, and entitled “A PLATFORM AND METHOD FOR ENGINEERING A HUMAN ORGANOID REPLICA FOR REPRODUCTIVE SCREENING,” the entirety of which is incorporated herein by reference.

Still referring to FIG. 1, databases, disclosed herein, may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Still referring to FIG. 1, platform 100 includes a computing device 104 configured to generate gene regulatory networks from transcriptomic datasets 120. Computing device 104 may include any computing device 104 as described in this disclosure, including without limitation a microcontroller, microprocessor, digital signal processor (DSP) and/or system on a chip (SoC) as described in this disclosure. Computing device 104 includes a processor 108 and a memory 112 communicatively connected to the processor 108, wherein memory 112 contains instructions configuring processor 108 generate gene regulatory networks. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device 104. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure. Computing device 104 may include, be included in, and/or communicate with a mobile device such as a mobile telephone or smartphone. Computing device 104 may include a single Computing device 104 operating independently or may include two or more computing devices 104 operating in concert, in parallel, sequentially or the like; two or more computing devices 104 may be included together in a single computing device 104 or in two or more computing devices 104. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices 104 in a first location and a second computing device 104 or cluster of computing devices 104 in a second location. Computing device 104 may include one or more computing devices 104 dedicated to data storage, security, distribution of traffic for load balancing, and the like. Computing device 104 may distribute one or more computing tasks as described below across a plurality of computing devices 104 of computing device 104, which may operate in parallel, in series, redundantly, or in any other manner used for distribution of tasks or memory 112 between computing devices 104. Computing device 104 may be implemented using a “shared nothing” architecture in which data is cached at the worker, in an embodiment, this may enable scalability of platform 100 and/or Computing device 104.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor 108 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, a “gene regulatory network (GRN),” as used in this disclosure, is a collection of molecular regulators that interact with each other and with other substances in the cell to govern the gene expression levels of mRNA and proteins. Gene regulatory network may determine the function of the cell. At the simplest level, regulation of gene expression may be characterized by binding of a transcription factor (TF) to a promoter region of the target gene and its concomitant activation or repression. “Transcription factors,” as used in this disclosure, are proteins involved in the process of converting, or transcribing, DNA into RNA. Transcription factors include a wide number of proteins, excluding RNA polymerase, which initiate and regulate the transcription of genes. Variation in responsiveness of a target gene to a TF, due to genetic variation, change in the environment or a combination thereof, can affect its expression and the resulting cellular phenotype. That said, gene expression is regulated by additional factor that affect gene expression (e.g., degradation). GRNs may help infer direct relationships among genes and provide a network-level analysis of biological function and importance. Differing network construction protocols, from supervised learning-based methods, model-based methods, and probabilistic graphs, can each possess inherent advantages and disadvantages, depending on the nature of the data being used. In some embodiments, computing device 104 may utilize a machine learning model, such as a classifier 126 to generate GRNS (i.e., TF-target gene-regulatory relationships) from transcriptomic datasets 120. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. Classifier 126 may be configured to output at least a datum that labels or otherwise identifies a set of data that are clustered together, found to be close under a distance metric as described below, or the like. Computing device 104 and/or another device may generate classifier 126 using a classification algorithm, defined as a processes whereby computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, the computational approaches for GRN generation may be broadly divided into two types: unsupervised, which may rely on availability of gene expression data, and supervised, which in addition to transcriptomics profiles may also use knowledge on known gene-regulatory interactions. The supervised approaches may be based on inductive reasoning to predict new interactions, whereby if one TF is known to regulate a gene, then all TF-gene pairs with similar features are likely to interact as well. To this end, the expression data profiles for a TF-gene pair may transform into feature vectors and provided as input to a supervised learning method. The learning method may be used to train the classifier configured to identify whether or not a pair of genes is involved in a regulatory interaction. Supervised learning approaches for GRN generation may be further grouped into local and global. In local approaches, classifier 126 may be configured to discriminate the target of each TF separately. Global approaches may use all TF-target gene pairs to train classifier 126 for gene-regulatory interactions. In some embodiments, classifier may be trained to output a gene regulatory graph 124. A “gene regulatory graph,” as used in this disclosure is a gene regulatory network graph containing a plurality of connected nodes representing transcription factors. In some embodiments, gene regulatory graph 124 may include a query-able connected graph structure, with only non-zero edges preserved. Gene regulatory graph 124 may be queried and or searched by data input. The input may by a plurality oogenesis RNA-seq transcriptomic datasets 120. Training data for classifier 126 may include sample models of GRN components and networks such as global features, local features, coupled ordinary differential equations, Boolean networks, Continuous networks, Stochastic gene networks and the like. In some embodiments, training data may include cell differentiation parameters such as epigenetic regulation, different time domains in response to external perturbation, hill coefficient, basal activity, decay rate, auto-activation, inflection point, self-inhibition strength, mutual inhabitation strength and the like. In some embodiments, training data may include gene regulatory networks of transcription factors as listed in Table. 1 below.

TABLE 1

| Transcription | Gene | |
|---|---|---|
| Factor | ID | Full Name |
| ZNF155 | 7711 | Zinc Finger Protein 155 |
| OTX2 | 5015 | Orthodenticle homeobox 2 |
| SOX13 | 9580 | SRY-box transcription factor 13 |
| DLX5 | 1749 | Distal-Less Homeobox 5 |
| ETV5 | 2119 | Ets variant 5 |
| ZNF502 | 91392 | Zinc Finger Protein 502 |
| SATB1 | 6304 | special AT-rich sequence-binding |
| | | protein-1 |
| LHX8 | 431707 | LIM Homeobox 8 |
| ZBTB39 | 9880 | Zinc Finger And BTB Domain |
| | | Containing 39 |
| KLF2 | 10365 | Kruppel Like Factor 2 |
| HHEX | 3087 | Hematopoietically-expressed |
| | | homeobox protein |
| SOHLH2 | 54937 | Spermatogenesis And Oogenesis |
| | | Specific Basic Helix-Loop-Helix 2 |

Still referring to FIG. 1, computing device 104 is configured to determine a candidate transcription factor 128. A “candidate transcription factor,” as used in this disclosure, is a transcription factor involved in general germline cell development. “Germline cell development,” as used in this disclosure, is the development of the cell lineage that gives rise to the reproductive cells, called gametes, of sexually reproducing organisms. Primordial germ cells are set aside in the early animal embryo, and divide and differentiate to produce sperm and egg, the male and female gametes. Candidate transcription factor 128 may include transcription factor families such as High Mobility Group Proteins (HMG), Paired box genes (PAX), GATA, Basic helix loop helix (bHLH), specificity proteins (Sp) family, forkhead box (FOX) family, HOX genes, ETS-domain TFs, steroid reproductive hormone receptors, zinc finger ZBTB proteins, with N-terminal BTB/POZ domains and the like. For example, candidate transcription factor 128 may include transcription factors HES1, HEY, HEY2, HAND1, HMGA1, HMGA2, Zf-C2H2, MYB, OU5F1, PHB, ZNF581, and the like. In some embodiments, computing device 104 may determine candidate transcription factor 128 based on a development need. For example, progenitor proliferation, cell migration, environmental control, and the like. Computing may utilize gene regulatory graph 124 to run metric calculations on the nodes representing transcription factors to identify the candidate set of factors to differentiate oocytes and spermatocytes at scale. A “metric calculation,” as used in this disclosure, is an algorithm used to model pairwise relations between items. “Spermatocytes” as used in this disclosure, are a type of male gametocyte in animals. A metric calculation may be used to link, group, and/or differentiate nodes in gene regulatory graph 124. For example, a metric calculation may include, Prim's algorithm, Kruskal's algorithm, Kosaraju's algorithm, Dijkstra's shortest path algorithm, and the like. In some embodiments, metric calculation may include “centrality”, which used herein, is algorithm that ranks nodes based on their connectivity. Connectivity may be correlated to the level of importance a transcription factor plays in cell differentiation into a particular cell type. For example, iPSCs into neurons, hepatocytes, and cardiomyocytes. Centrality algorithms, specifically tailored for time-series RNA-seq data, may be developed to apply to these nodes. “Time-series data,” as used in this disclosure, is a sequence of data points collected over time intervals. In some embodiments, the centrality algorithm may be incorporated into a machine learning model configured to intake gene regulatory graph 124 and output the ranked nodes. Ranking may be established by categories, such important, irrelevant, indifferent, and the like. Training data may transcription factors involved in cell differentiation, transcription factors effective in cell type maturation, and the like.

Still referring to FIG. 1, computing device 104 may be configured to generate classifier 126 using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(AB) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate classifier 126 using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing classifier 126 to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where a is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, computing device 104 is configured analyze the impact of candidate transcription factor 128 in germline cell development. This may include analyzing the necessity of the candidate transcription factors 128 in correlation to essential transcription factors in germ cell differentiation of a particular cell type. For example, transcription factors SOX17, TFAP2C, and BLIMPL are necessary for differentiation of human primordial germ cell-like cells (hPGCLCs), the precursors of oocytes and spermatocytes. hPGCLCs may be generated from iPSCs. To establish necessity, computing device 104 may utilize Clustered Regularly Interspaced Short Palindromic Repeats (CRISPR) technology 130. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CR ISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of IN vitro Transcription (SPRINT).

Still referring to FIG. 1, in some embodiments, CRISPR-mediated knockdown of candidate transcription factors 128 may be performed in human iPSCs in conjunction with in vitro protocols to generate hPGCLCs. “Gene knockdown,” as used in this disclosure is a technique in which the expression of one or more of an organism's genes is reduced. The reduction, also referred to as repression in this disclosure, may occur either through genetic modification or by treatment with a reagent such as a short DNA or RNA oligonucleotide that has a sequence complementary to either gene or an mRNA transcript. “CRISPR-mediated knockdown,” as used in this disclosure, is the use of CRISPR technology to execute a gene knockdown technique. In some embodiments, CRISPR-mediated knockdown may include CRISPRi: CRISPR interference, using dCas9, without additional proteins. In some embodiments, CRISPR-mediated knockdown may include CRISPRi: CRISPR interference, using dCas9, in combination with other proteins. In some embodiments, CRISPR-mediated knockdown may include Cas13 family enzymes. In some embodiments, in vitro cell differentiation of pluripotent cells may include protocols as disclosed in U.S. Nonprovisional application Ser. No. 17/846,725, filed on Jun. 22, 2022, and entitled “APPARATUS AND METHOD FOR INDUCING HUMAN OOCYTE MATURATION IN VITRO,” the entirety of which is incorporated herein by reference. Based on the results, it may be determined if a candidate transcription factor 128 is a positive or negative regulator for hPGCLC formation and whether it is necessary for hPGCLC differentiation. For example, CRISPR-mediated knockdown of candidate transcription factors 128 may be used to identify DLX5, HHEX, and FIGLA transcription factors whose individual overexpression drives potent enhancement of hPGCLC formation. In this example, platform 100 may be used to demonstrate that DLX5 overexpression rescues loss of BMP4 during germ cell formation. Furthermore, phenotypic assays of cell migration, cell morphology, cell signaling, as well as epigenetic analysis may utilized to probe the likely role of the candidate factor in germ cell development. Results of the assay may be validated by homozygous knockouts of each transcription factor. The data may then elucidate the core transcriptional regulation that drives germline cell development, while also identifying factors that can have critical intermediate effects. Still referring to FIG. 1, computing device 104 is configured to output a set of critical transcription factors 132. A “critical transcription factor,” as used in this disclosure, is a transcription factor whose multiplexed overexpression and repression directs iPSC differentiation. “Multiplex gene expression (MGE),” as used in this disclosure is an analysis that provides direct and quantitative measurement of multiple endogenous mRNAs using a multiplexed detection system coupled to reverse transcription-PCR. For example, multiplex methods may include, real-time multiplex PCR, multiplex assay, and the like. “Overexpression,” as used in this disclosure is the excessive expression of a gene. “Repression,” as used in this disclosure is the recessive expression of a gene. For example, critical transcription factors 132 may include the overexpression or repression of GATA4, MEF2C, TBXS, ESRRG, MESP1, and the like. Computing device 104 may identify critical transcription factors 132 by utilizing a human iPSC (hiPSC) line harboring stable integration of CRISPR transcriptional activators and repressors. In one embodiment, computing device 104 may identify critical transcription factors 132 by utilizing a hiPSC line harboring stable integration of complementary DNA (cDNA) overexpression construct. A “human iPSC line,” as used in this disclosure is a collection of iPSC cells. A “CRISPR transcriptional activator” as used in this disclosure, is a cell complex, derived using CRISPR technology, containing transcription factors that increases transcription of a gene or set of genes. A “CRISPR transcriptional activator” as used in this disclosure, is a cell complex, derived using CRISPR technology, containing transcription factors that prevent transcription of a gene or set of genes. As used in this disclosure, a “cDNA” is DNA synthesized from a single-stranded RNA template in a reaction catalyzed by the enzyme reverse transcriptase. An “overexpression,” as disclosed herein, is excessive expression of a gene caused by increased frequency of transcription. In some embodiments, a multiplexed, high throughput screen of 50 candidate transcription factors 128 may performed utilizing LentiArray and LentiPool gRNA libraries for CRISPR screening. For example, using a library of lentiviruses that each express 1-6 sgRNAs transduced into the iPSC line, to determine which single guide RNA (sgRNA) sequences and therefore which candidate transcription factors 128 may drive differentiation of germ cell-like cells or earlier intermediates. Through multiple rounds of library refinement, a minimal set of sgRNAs that modulate the expression of up to 6 factors may be determined that is sufficient for driving germ cell differentiation. The CRISPR-derived germ cell-like cells may be compared to the profiles of mature germ cells and their intermediates via single-cell RNA-seq, proteomic, and morphology analysis to determine the physiological similarity between CRISPR-derived germ cell-like cells and mature germ cells (and intermediates of mature germ cells).

Still referring to FIG. 1, in some embodiments, CRISPR 130 may be used to perform a pooled CRISPR screen utilizing RNA libraries and/or datasets as described above. In a “pooled CRISPR screen,” as used herein, various genetically encoded perturbations are introduced into pools of cells. The targeted cells proliferate under a biological challenge such as cell competition, drug treatment or viral infection. Subsequently, the perturbation-induced effects are evaluated by sequencing-based counting of the guide RNAs that specify each perturbation. The typical results of such screens may be ranked lists of genes that confer sensitivity or resistance to the biological challenge of interest. Contributing to the broad utility of CRISPR screens, adaptations of the core CRISPR technology may make it possible to activate, silence or otherwise manipulate the target genes. Moreover, high-content read-outs such as single-cell RNA sequencing and spatial imaging may help characterize screened cells with unprecedented detail.

Still referring to FIG. 1, a plurality of algorithms as described in this disclosure may be applied in CRISPR 130 for CRISPR knockout, activation, inactivation, pooling screens, and the like. For example, “redundant siRNA activity (RSA),” which as used herein, is designed to identify important genes in RNA interference (RNAi) loss-of-function screens. RSA works by initially ranking all targeting guides by decreasing log fold change between the initial condition and final condition. The algorithm then assigns a p value to each gene using an iterative hypergeometric distribution formula that measures the statistical significance of a gene having highly ranked guides, assuming that under the null distribution, the ranks are uniformly distributed. Only the rankings of the guides, not the magnitude of the log fold change, are used in computing the p value. This approach allows for rare off-target guides with high effect sizes to be deprioritized compared to guides that all perform around the same. As output, RSA returns an ordering of genes ranked by essentiality but not their associated p values. In another example, CRISPR 130 may use “barcode-sequencing,” which as used herein, is a next-generation sequencing (NGS) technique that reads genome-integrated artificial sequences called barcodes that specifically mark biological materials, such as cells or genes, with unique sequences.

Still referring to FIG. 1, in some embodiments computing device 104 may be configured to develop highly predictive CRISPRa and CRISPRi tools utilizing deep learning models for sgRNA selection, may include a plurality of deep learning-based architectures. A used in this disclosure, a “deep learning model,” is a type of machine learning based on artificial neural networks (described further below) in which multiple layers of processing are used to extract progressively higher-level features from data. For example, a deep learning model may include a model with only fully connected layers (a fully connected neural network—FCNN), a model with convolutional layers (a convolutional neural network—CNN), and a model with recurrent long-short term memory layers (an LSTM model).

Still referring to FIG. 1, validation of critical transcription factors 132 and/or the CRISPR-mediated high-throughput screening platform on iPSCs for targeted differentiation as described in this disclosure, may include comparing critical transcription factors 132 to base transcription factors using a plurality of methods. For example, a comparison method may include immunofluorescence staining. “Immunofluorescence (IF),” as used in this disclosure, is an immunochemical technique that allows detection and localization of a wide variety of proteins. IF allows for excellent sensitivity and amplification of signal in comparison to immunohistochemistry, employing various microscopy techniques. For example, immunofluorescence staining may be used to confirm that an overexpression of critical transcription factor 132 exhibits nominal protein expression hallmarks of conventional transcription factors that drive iPSC differentiation. In some embodiments, method may include epigenetic profiling using enzymatic methylation sequencing techniques. In some embodiments, method may include “CUT&RUN sequencing,” which as used herein, is a method used to analyze protein interactions with DNA. CUT&RUN sequencing may provide low levels of background signal because of in situ profiling which retains in vivo 3D confirmations of transcription factor-DNA interactions.

Still referring to FIG. 1, in some embodiments validation may include comparison to transcriptomic datasets 120 and/or RNA-seq datasets from biological databases as described throughout this disclosure for phenotype analysis. For example, an atlas of 100 deposited RNA-seq FASTQ files may be curated from various studies, where ovarian somatic and germ cells may be obtained or derived from human samples, further analyzed, and deposited. The atlas may include granulosa cell data at various stages of fetal and adult ovarian development, as well as oogenesis data from stem cells through primordial germ cell specification, and finally to oogonia and oocyte from various stages of follicular development. In another example, raw data files alongside collected RNA-Seq datasets may be aligned to the latest build of the human reference genome (GRCh38) utilizing the Spliced Transcripts Alignment to a Reference (STAR) alignment tool, to construct count matrices aligning sequencing reads to the known set of human genes. A standard DESeq2 analysis package in R may be used to estimate variance-mean dependence in count data, and subsequently calculate differential expression of each gene for every sample utilizing a negative binomial distribution.

Still referring to FIG. 1, validation may also include, a “Transcriptome Overlap Measure (TROM),” which as used herein, is a method to identify associated genes that capture molecular characteristics of biological samples and subsequently comparing the biological samples by testing the overlap of their associated genes. TROM scores may be calculated as the −log 10(Bonferroni corrected p value of association) on a scale of 0-300. The TROM magnitude may be positively correlated with similarity between two independent samples, with a standard threshold of 12 as an generally-accepted indicator of significant similarity.

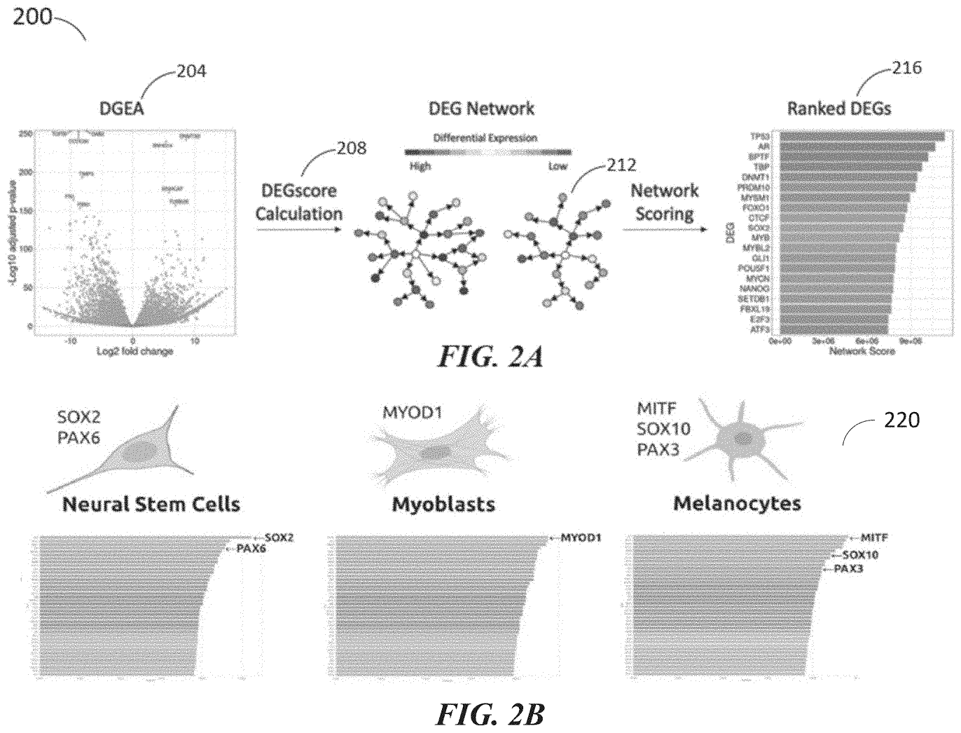

Referring now to FIG. 2A, in some embodiments, a metric calculation method 200 may include a differentially expressed gene (DEG) network analysis (DGEA) 204 to analyze the impact of the candidate transcription factor in germline cell development. “DEG network analysis 204,” as used herein, is a scoring method utilizing transcriptomic data from a starting cell state and a target cell state. DGEA 204 may be performed to determine significant gene expression changes. A DEG score 208 may be generated for each gene by combining the traditional DEG metrics (fold-change, p value) with cell phenotype information (correlation with desired phenotype). To infer phenotype causality as well as identify DEGs with small changes but potentially large effects, a layer of protein network connectivity 212 may be added to DEG scoring. Transcriptomic dataset database 116 and gene regulatory graph 124 as described in accordance with FIG. 1, biological databases (i.e., STRING interaction database), and other web resources of known and predicted protein—protein interactions may be utilized to traverse each DEG's protein network 212 and calculate a score that combines its DEG score 208 with the degree of connectivity. As a result, computing device 104 as described in accordance with FIG. 1 may output a list preferentially ranked DEGs 216 with large significant changes between the two cell states that are also highly connected to other highly differentially expressed DEGs.

Referring now to FIG. 2B, in one embodiment, a validation 220 regarding a prediction algorithm 224 of central TFs in known differentiation protocols using graph theory-based TF discovery pipeline is illustrated. Validation 220 may be performed using existing RNA-seq datasets of neuronal stem cell, myoblast, and melanocyte differentiation. Experimentally validated TFs are demonstrating predictive capability of the pipeline. In order to provide an algorithm that may be highly sensitive to small intermediary transcriptomic changes across time-series and may overcome the dependency on the availability of protein interaction data, time-series transcriptomic data may be combined with graph theory-based centrality analysis. In one embodiment, stochastic gradient boosting machines may be utilized to train GRNs and calculate a PageRank of each genetic factor post network construction and graph pruning. In one embodiment, a normalized fold-change representation for each gene at different stages of the cell state conversion may be required. Compared with traditional DEG approaches, validation 220, in one embodiment, demonstrates that the prediction algorithm 224 may effectively identify known experimentally validated causal regulators within the predicted top factors.

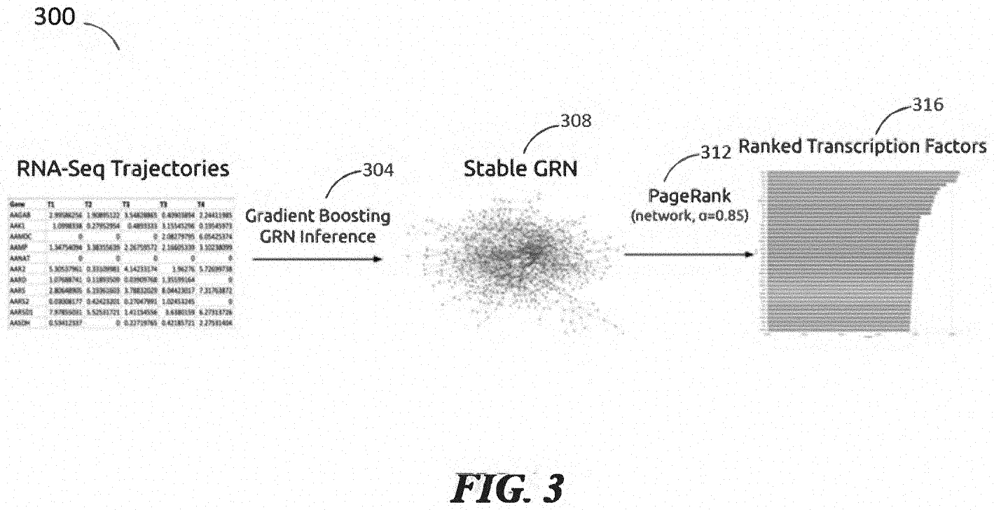

Referring now to FIG. 3, in one embodiment, a GRN centrality analytic algorithm 300 may be performed to reduce a DEGA's inherent dependency on the availability of protein interaction data and increase the sensitivity to small intermediary transcriptomic changes across time-series data. GRN centrality analytic algorithm 300, in one embodiment, combines time-series transcriptomic data with graph theory-based centrality analysis by utilizing stochastic gradient boosting machines 304 to train GRNs 308 and calculate a PageRank 312 of each genetic factor post network construction and graph pruning. In one embodiment, and without limitation, the algorithm 300 requires a normalized fold-change representation for each gene at different stages of the cell state conversion and generates a graphical representation of ranked transcription factors 316 with the highest global importance.

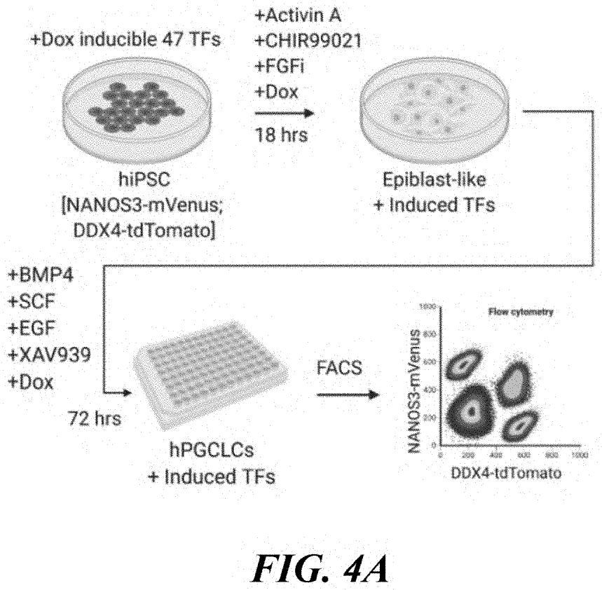

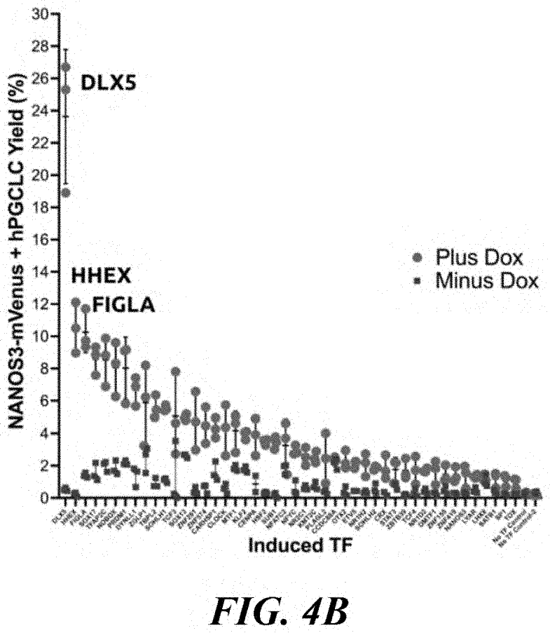

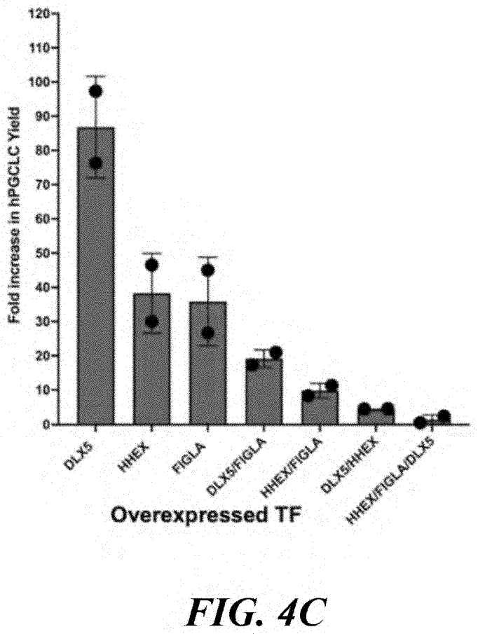

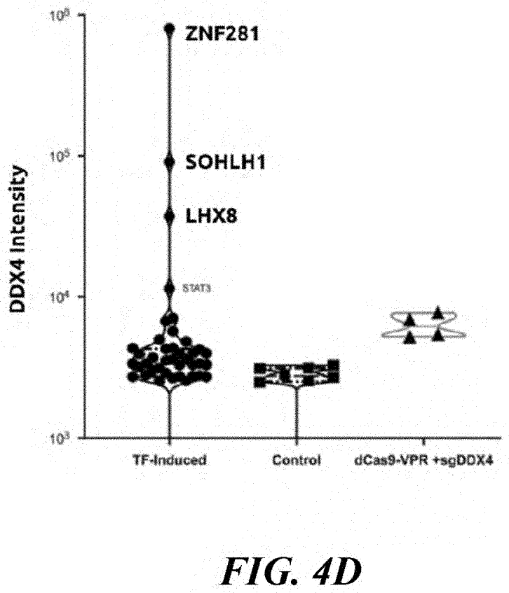

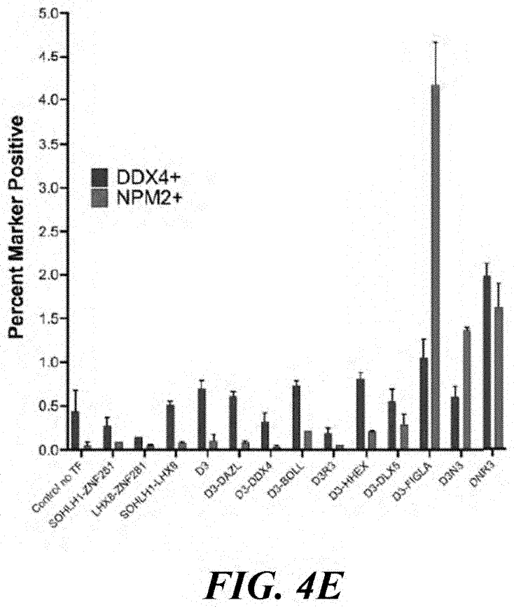

Referring now to FIGS. 4A-E, in one embodiment, a characterization of the contribution of 47 TFs to germ cell and oogonia formation via cDNA overexpression screening are illustrated. in one embodiment, doxycycline-inducible vectors expressing a full-length cDNA may be generated for each of 47 TFs identified by a TF prediction algorithm. Each vector harbored a 50 bp barcode on the 3′ UTR of the cDNA and may be piggyBac integratable. In one embodiment, a NANOS3-mVenus may be constructed; DDX4-tdTomato dual reporter hiPSC line (N3VD4T) using CRISPR-Cas9-mediated homology directed repair (HDR), and 47 hiPSC lines may be generated harboring integrations of each TF individually through super piggyBac transposase-mediated insertion. In one embodiment, polyclonal pools for each TF may be utilized for screening purposes.

Referring now to FIG. 4A, in one embodiment, a monolayer induction protocol 404 may be deployed, wherein hPGCLCs may be induced through epiblast-like intermediates followed by BMP4 induction for 4 days in a monolayer condition. In one embodiment, the monolayer induction protocol 404 may be optimized by eliminating vitamin A and increasing Activin A concentration to increase hPGCLC yield.

Referring now to FIG. 4B, in one embodiment, monolayer induction protocol 404 may be utilized to assess NANOS3+hPTCLC yield via flow cytometry in the presence or absence of doxycycline for the 47 TFs in triplicate. FIG. 4B illustrates that all 47 TFs drive upregulation of NANOS3+hPGCLC yield, which highlights the general utility of the TF prediction algorithm for identifying TF regulators of human germline development. For instance, 3TFs (DLX5, HHEX, and FIGLA) induced a NANOS3+ yield higher than that of three known TF regulators of hPGCLC development: SOX17, TFAP2C, and PRDM1. In one embodiment, a contribution of DLX5, HHEX, and FIGLA to hPGCLC formation may be further elucidated, wherein an overexpression of DLX5 may be able to replace exogenous BMP4 in the induction of hPGCLCs, driving potent hPGCLC formation in the absence of BMP4. In one embodiment, as quantified by both the NANOS3 reporter and CD38 cell surface marker expression, overexpression of DLX5, HHEX, and FIGLA may increase hPGCLC formation in both floating aggregate and monolayer cultures.

Referring now to FIG. 4C, combinatorial overexpression of DLX5, HHEX, and FIGLA may exhibit lower hPGCLC yield compared to individual overexpression and/or combinatorial overexpression of DLX5/FIGLA and/or combinatorial overexpression of HHEX/FIGLA and/or combinatorial overexpression of DLX5/HHEX.

Referring now to FIG. 4D, DDX4+ oogonia-like yield via flow cytometry in the presence or absence of doxycycline for 47 TFs in triplicate is assessed. Compared to control, the overall percentage of DDX4+ cells may not greatly enriched by any single TF. However, a small percentage of cells with elevated DDX4+ expression may be identified in the ZNF281, LHX8, and SOHLH1 induction conditions.

Referring now to FIG. 4E, an induction of a large percentage of DDX4+ cells based on the overexpression of all three TFs is illustrated. The high DDX4+ population may be obtained in just 4 days in monolayer through direct TF induction during hPGCLC formation. NPM2 is a critical oocyte marker gene, involved in chromatin organization. In one embodiment, employing a DDX4-tdTomato; NPM2-mGreenLantern reporter hiPSC line (D4TP2G), an addition of other TFs and RNA-binding proteins, including DLX5, HHEX, FIGLA, DAZL, DDX4, and BOLL, to the combinatorial overexpression of ZNF281, LHX8, and SOHLH1 may increase the DDX4+ yield. For instance, and without limitation, the addition of FIGLA may drive an increase in DDX4+ yield and NPM2+ yield. In another embodiment, the addition of all TFs—ZNF281, LHX8, SOHLH1, DLX5, HHEX, FIGLA, DAZL, DDX4, and BOLL—may induce robust DDX4+ yield and a modest NPM2+ yield. In one embodiment, overexpression of ZNF281, SOHLH1, and LHX8 individually or in combination during hPGCLC differentiation with the addition of FIGLA, HHEX, DLX5, DAZL, BOLL, and DDX4 may increase DDX4+ yield.

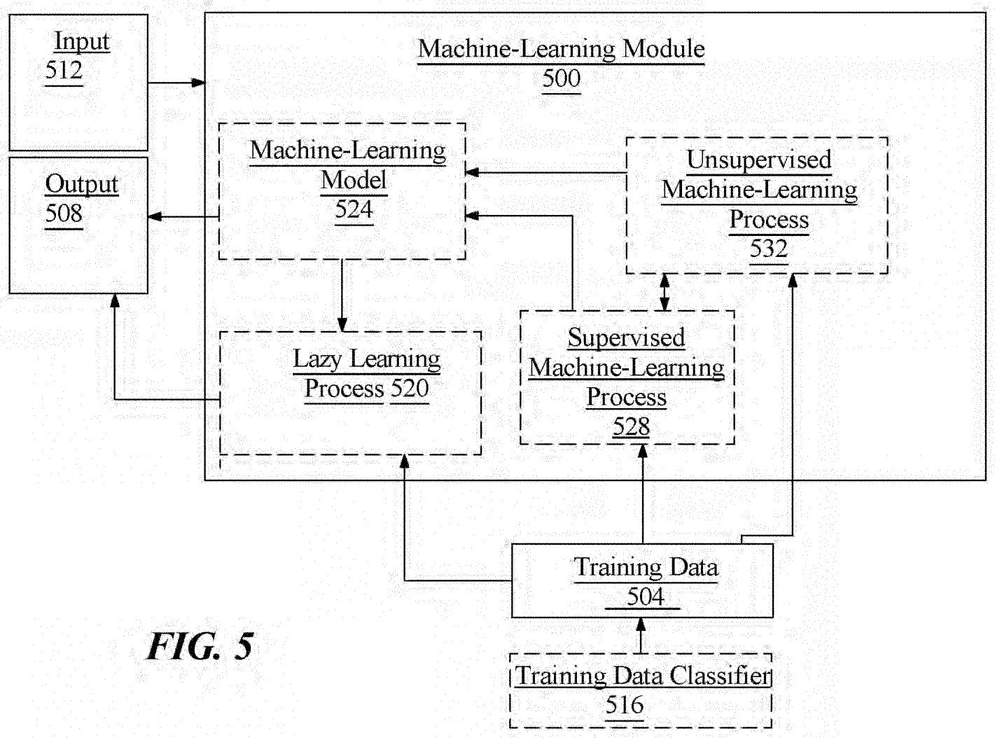

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device 104/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a processes whereby a computing device 104 and/or any module and/or component operating thereon derives a classifier from training data 504. Classification may be performed using, without limitation, linear classifiers such as without limitation logistic regression and/or naïve Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory 112; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs and outputs, as described above, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the LASSO model is the Frobenius norm amounting to the square root of the sum of squares of all terms. Linear regression models may include the elastic net model, a multi-task elastic net model, a least angle regression model, a LARS LASSO model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

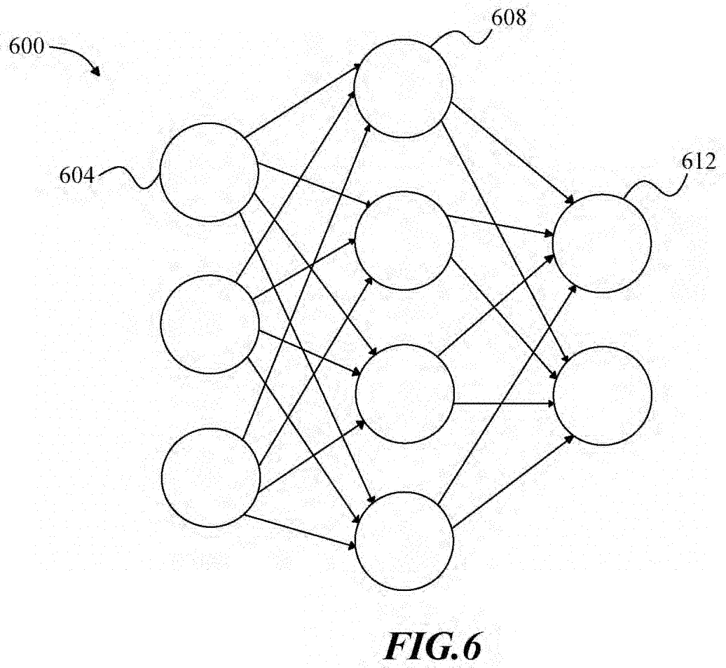

Referring now to FIG. 6, an exemplary embodiment of neural network 600 is illustrated. A neural network 600 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 604, one or more intermediate layers 608, and an output layer of nodes 612. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

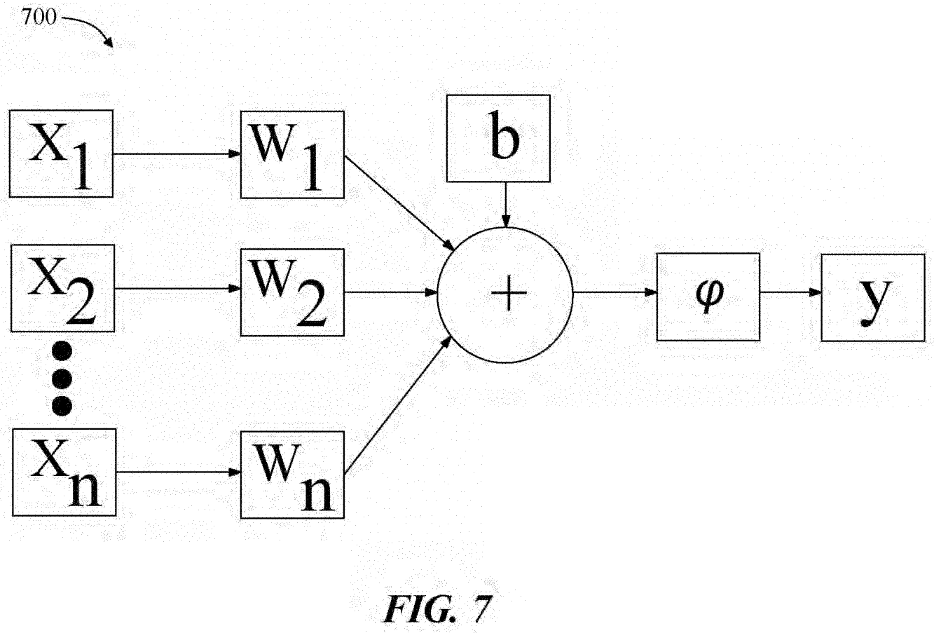

Referring now to FIG. 7, an exemplary embodiment of a node of a neural network is illustrated. A node may include, without limitation a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight.

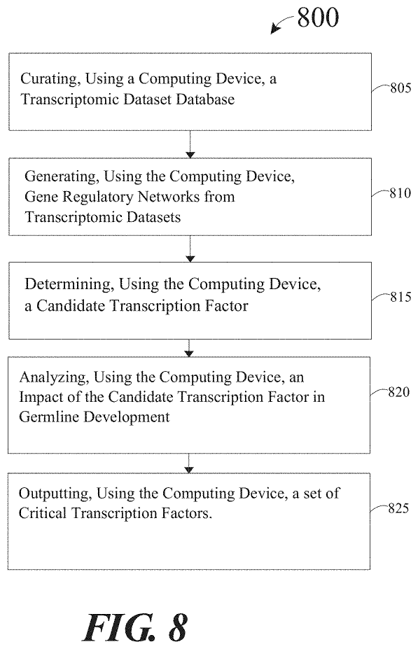

Referring now to FIG. 8, is an exemplary flow diagram of a method for determining critical transcription factors for in vitro germ cell differentiation. Method 800 may utilize a computing device as described in FIGS. 1-7. At step 508, method 800 includes curating, using a computing device, a transcriptomic dataset database, this may be implemented as disclosed with reference to FIGS. 1-7. In some embodiments, curating the transcriptomic dataset database may include generating, using the computing device, an integrated normalized database comprising RNA-seq data. At step 810, method 800 include generating, using the computing device, gene regulatory networks from transcriptomic datasets, this may be implemented as disclosed with reference to FIGS. 1-7. In some embodiments, generating, using the computing device, the gene regulatory networks may include utilizing a machine-learning model configured to output a gene regulatory graph.

Still referring to FIG. 8, at step 518, method 800 includes determining, using the computing device, a candidate transcription factor, this may be implemented as disclosed with reference to FIGS. 1-7. In some embodiments, determining, using the computing device, a candidate transcription factor may include analyzing a gene regulatory graph to identify a critical set of transcription factors to differentiate oocytes and spermatocytes. Additionally, identifying a critical set of transcription factors may include utilizing a machine-learning model to generate a metric calculation as a function of the gene regulatory graph. The metric calculation may include a centrality algorithm, wherein the centrality algorithm is configured for time-series RNA-seq data.

Still referring to FIG. 8, at step 820, method 800 includes analyzing, using the computing device, an impact of the candidate transcription factor in germline cell development, this may be implemented as disclosed with reference to FIGS. 1-7. In some embodiments, analyzing, using the computing device, the impact of the candidate transcription factor further comprises CRISPR-mediated knockdown of candidate transcription factors. The set of critical transcription factors may include multiplexed overexpression and repression direct iPSC differentiation.

Still referring to FIG. 8, at step 825, method 800 includes outputting, using the computing device, a set of critical transcription factors, this may be implemented as disclosed with reference to FIGS. 1-7. In some embodiments, outputting the set of critical transcription factors may include utilizing a human iPSC line harboring stable integration of CRISPR transcriptional activators and repressors.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

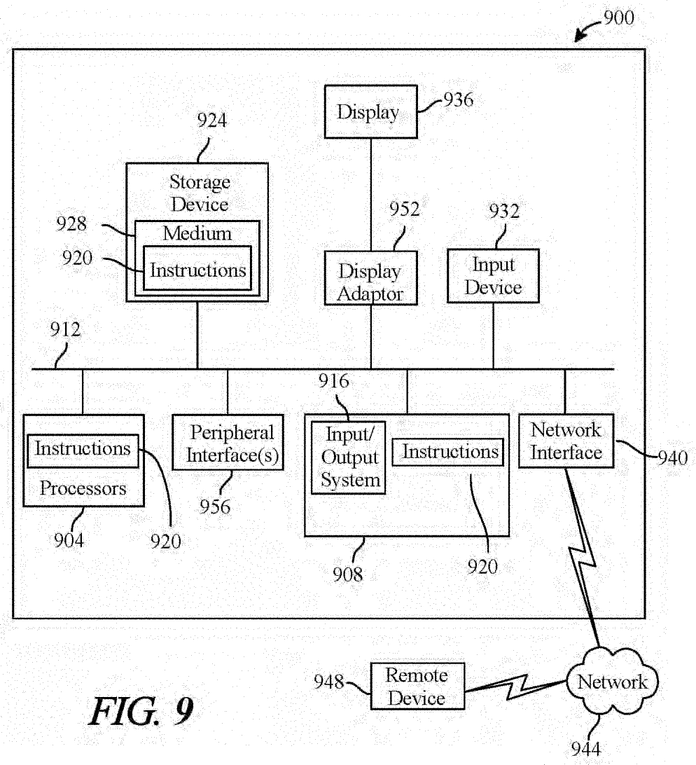

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, platforms, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A platform for determining critical transcription factors for TF-based hiPSC differentiation, the platform comprising:
a transcriptomic dataset database;
at least a processor; and
a memory communicatively connected to the processor, the memory containing instructions configuring the at least a processor to:
generate a plurality of gene regulatory networks from a plurality of transcriptomic datasets;
determine a candidate transcription factor;
analyze an impact of the candidate transcription factor in germline cell development; and
output a set of critical transcription factors.

2. The platform of claim 1, wherein the transcriptomic dataset database comprises an integrated normalized database comprising RNA-seq data.

3. The platform of claim 1, wherein generating the gene regulatory networks further comprise utilizing a machine-learning model configured to output a gene regulatory graph.

4. The platform of claim 1, wherein determining a candidate transcription factor comprises analyzing a gene regulatory graph to identify the set of critical transcription factors to differentiate oocytes.

5. The platform of claim 4, wherein identifying the set of critical transcription factors further comprises utilizing a machine-learning model to generate a metric calculation as a function of the gene regulatory graph.

6. The platform of claim 5, wherein the metric calculation comprises a criticality algorithm.

7. The platform of claim 6, wherein the criticality algorithm is configured for time-series RNA-seq data.

8. The platform of claim 1, wherein analyzing the impact of the candidate transcription factor comprises CRISPR-mediated knockdown of candidate transcription factors.

9. The platform of claim 1, wherein the set of critical transcription factors comprises transcription factors exhibiting multiplexed overexpression and repression that directs iPSC differentiation.

10. The platform of claim 1, wherein outputting the set of critical transcription factors further comprises utilizing a human iPSC line harboring stable integration of CRISPR transcriptional activators and repressors.

11. The platform of claim 1, wherein outputting the set of critical transcription factors further comprises utilizing a human iPSC line harboring stable integration of cDNA overexpression constructs.

12. A method for determining critical transcription factors for TF-based hiPSC differentiation, the method comprising:
curating, using a computing device, a transcriptomic dataset database;
generating, using the computing device, gene regulatory networks from a plurality of transcriptomic datasets;
determining, using the computing device, a candidate transcription factor;
analyzing, using the computing device, an impact of the candidate transcription factor in germline cell development; and
outputting, using the computing device, a set of critical transcription factors.

13. The method of claim 12, wherein curating the transcriptomic dataset database comprises generating, using the computing device, an integrated normalized database comprising RNA-seq data.

14. The method of claim 12, wherein generating, using the computing device, the gene regulatory networks further comprise utilizing a machine-learning model configured to output a gene regulatory graph.

15. The method of claim 12, wherein determining, using the computing device, a candidate transcription factor comprises analyzing a gene regulatory graph to identify the set of critical transcription factors to differentiate oocytes.

16. The method of claim 15, wherein identifying the set of critical transcription factors further comprises utilizing a machine-learning model to generate a metric calculation as a function of the gene regulatory graph.

17. The method of claim 16, wherein the metric calculation comprises a criticality algorithm.

18. The method of claim 17, wherein the criticality algorithm is configured for time-series RNA-seq data.

19. The method of claim 12, wherein analyzing, using the computing device, the impact of the candidate transcription factor further comprises utilizing CRISPR-mediated knockdown of candidate transcription factors.

20. The method of claim 12, wherein the set of critical transcription factors comprises transcription factors exhibiting multiplexed overexpression and repression that directs iPSC differentiation.

21. The method of claim 12, wherein outputting, using the computing device, the set of critical transcription factors further comprises utilizing a human iPSC line harboring stable integration of CRISPR transcriptional activators and repressors.

22. The platform of claim 1, wherein outputting the set of critical transcription factors further comprises utilizing a human iPSC line harboring stable integration of cDNA overexpression constructs.
